(19) United States
(12) Patent Application Publication
(10) Pub. No.: US 2003/0082116 A1
(43) Pub. Date: May 1, 2003

(54) Adhesive compositions containing dual function stabilizers and active agents

(75) Inventors: Ibraheem Badejo, Morrisville, NC (US); Anthony Sherbondy, Raleigh, NC (US); Timothy McAfoos, Wooster, OH (US)

Correspondence Address: OLIFF & BERRIDGE, PLC, P.O. BOX 19928, ALEXANDRIA, VA, 22320, US

(73) Assignee: CLOSURE MEDICAL CORPORATION, Raleigh, NC (US)

(21) Appl. No.: 09/964,415

(22) Filed: Sep. 28, 2001

(51) Int. Cl. A61K 7/26; A61K 35/78

(52) U.S. Cl. 424/58; 523/120; 424/728; 424/730; 424/764; 424/765; 424/745; 424/748; 424/770; 424/736; 424/757; 424/754

(57) ABSTRACT

An adhesive composition includes a polymerizable adhesive monomer and at least one dual function stabilizer. The stabilizer provides superior stabilization and shelf-life of the composition, and enhances wound healing properties of the monomer composition.

### BACKGROUND OF THE INVENTION

1. Field of Invention

The present invention relates to monomer and polymer adhesive and sealant compositions, and to their production and use for industrial and medical applications. In particular, the present invention relates to the incorporation of dual function agents in adhesive compositions, such as to provide combined stabilization properties to the composition and active agent properties to the application surface such as to enhance the wound healing properties of such compositions when used for medical purposes.

2. Description of Related Art

Monomer and polymer adhesives are used in both industrial (including household) and medical applications. Included among these adhesives are the 1,1-disubstituted ethylene monomers and polymers, such as the α-cyanoacrylates. Since the discovery of the adhesive properties of such monomers and polymers, they have found wide use due to the speed with which they cure, the strength of the resulting bond formed, and their relative ease of use. These characteristics have made the α-cyanoacrylate adhesives the primary choice for numerous applications such as bonding plastics, rubbers, glass, metals, wood, and, more recently, biological tissues.

It is known that monomeric forms of α-cyanoacrylates are extremely reactive, polymerizing rapidly in the presence of even minute amounts of an initiator, including moisture present in the air or on moist surfaces such as animal (including human) tissue. Monomers of α-cyanoacrylates are anionically polymerizable or free radical polymerizable, or polymerizable by zwitterions or ion pairs to form polymers. Once polymerization has been initiated, the cure rate can be very rapid.

Medical applications of 1,1-disubstituted ethylene adhesive compositions include use as an alternate or an adjunct to surgical sutures and/or staples in wound closure, as well as for covering and protecting surface wounds such as lacerations, abrasions, burns, stomatitis, sores, minor cuts and scrapes, and other wounds. When an adhesive is applied to surfaces to be joined, it is usually applied in its monomeric form, and the resultant polymerization gives rise to the desired adhesive bond.

The industrial production of 1,1-disubstituted ethylene monomer adhesive compositions has been optimized to provide adhesives with rapid cure rates and high bond strengths. However, the desire to provide an adhesive with a rapid cure rate has led to problems with shelf-life. The shelf-life of these adhesives is primarily related to stability (i.e., constancy of compositional nature), uncured physical properties, rate of cure of the adhesive, as well as final cured properties of the composition. For example, the shelf-life of a monomeric α-cyanoacrylate composition is related to the amount of time the composition can be stored before unacceptable levels of polymerization occur. Unacceptable levels are indicated by a level of polymerization product that reduces the usefulness of the composition in the application for which it is produced. It is well known that monomeric forms of α-cyanoacrylates polymerize rapidly in the presence of even minute amounts of an initiator, and that once polymerization has been initiated, the rate of cure can be very rapid. Therefore, in order to obtain a monomeric α-cyanoacrylate composition with an extended shelf-life, polymerization inhibitors such as anionic and free radical stabilizers are often added to the compositions. However, addition of such stabilizers can result in substantial retardation of the cure rate of the composition. Therefore, in the production of industrial α-cyanoacrylate adhesives, the amount of stabilizers added is minimized so that the cure rate is not adversely affected.

Cyanoacrylate adhesives used in medical applications preferably have a shelf-life of at least twelve months. In order to achieve a useful shelf-life, anionic and free-radical stabilizers are generally added to the monomer compositions.

As disclosed in U.S. Pat. Nos. 3,559,652 to Banitt et al. and 5,582,834 to Leung et al., for example, suitable stabilizers for medically useful α-cyanoacrylate compositions include Lewis acids such as sulfur dioxide, nitric oxide, and boron trifluoride, as well as free-radical stabilizers including hydroquinone, monomethyl ether hydroquinone, nitrohydroquinone, catechol, and monoethyl ether hydroquinone. The combination of the two anionic stabilizers sulfur dioxide and sulfonic acid is also known and is disclosed in, for example, British Patent Application GB 2 107 328 A. However, the use of these two anionic stabilizers in combination does not overcome the “speed loss” seen in other 1,1-disubstituted ethylene adhesive compositions.

In addition to having an extended shelf-life, cyanoacrylate compositions for use in many medical applications should be sterile. Due to the importance of achieving and maintaining sterility of these compositions, when an additive, such as an anionic or free-radical stabilizer, is added to an α-cyanoacrylate composition, it should be added prior to sterilization. However, regardless of the type and number of additives, sterilization of α-cyanoacrylate adhesive compositions is often difficult to achieve. For example, widely practiced methods of sterilization, such as heat sterilization and ionizing radiation, are often not suitable for use with monomeric cyanoacrylate compositions. Problems arise due to polymerization of the monomer during the sterilization process, even in the presence of stabilizers. In many cases, sterilization-induced polymerization is so severe that the resulting product is unusable. Furthermore, even when the sterilized product is still useable, the shelf-life at desired storage temperatures, such as under refrigerated conditions or at room temperature, can be shortened to such a degree that the product is not suitable for commercialization.

Methods currently used to package and sterilize α-cyanoacrylate monomer compositions have been developed with the recognition that, to improve efficiency and productivity, the packaging and sterilizing steps should be performed in rapid succession. However, these methods do not provide the desired shelf-life of the adhesive compositions in all packaging materials.

Furthermore, during sterilization, much or all of the stabilizer can be consumed or converted to another compound. For example, U.S. Pat. No. 5,530,037 to McDonnell et al. discloses that when a low level of sulfur dioxide is used to stabilize a cyanoacrylate composition, all of the sulfur dioxide is converted to sulfuric acid during the sterilization process. Thus, although polymerization during sterilization can be minimized by use of low levels of sulfur dioxide, and shelf-life of he sterilized α-cyanoacrylate adhesive composition can be increased, shelf-life might be improved by the presence of increased amounts of sulfur dioxide in the initial composition. Unfortunately, at initial high levels of a stabilizer, the general performance of the adhesive can be impaired and the shelf life provided still is less than desired.

McDonnell et al. also teaches that the use of the free radical stabilizers butylated hydroxyanisole (BHA) and butylated hydroxytoluene (BHT), in combination with 100 parts per million (final concentration) sulfur dioxide, are not effective at stabilizing α-cyanoacrylate compositions during gamma irradiation sterilization. Rather, they must be present in concentrations substantially above 1000 parts per million or higher in order to provide effective stabilization.

In addition, various phenolic stabilizers have been used for stabilizing adhesive compositions. For instance, McDonnell et al. teaches the use of a free radical stabilizer selected from phenolic antioxidants (except for hydroquinone). U.S. Pat. No. 4,125,494 to Schoenberg teaches the use of anionic inhibitors including phenolic compounds such as hydroquinone, t-butyl catechol, pyrocatechol, p-methoxyphenol, and the like. U.S. Pat. No. 4,724,177 to Russo teaches the use of free radical stabilizers including hydroquinone, monomethylether hydroquinone, nitrohydroquinone, and hydroquinone monoethylether. U.S. Pat. No. 5,034,456 to Katsumura teaches the use of radical polymerization inhibitors including hydroquinone, hydroquinone monomethyl ether, catechol, pyrogallol and the like. U.S. Pat. No. 4,321,180 to Kimura teaches the use of radical polymerization inhibitors such as aryl alcohols, phenol, cresols, hydroquinone, benzoquinone, α-naphthol, β-naphthol, catechol, pyrogallol, bisphenol-A, bisphenol-S, 2,6-di-tert-butylphenol, 2,6-di-tert-butylcresol, 2,2′-methylene-bis(4-methyl-6-tert-butylphenol), 4,4′-butylidene-bis(3-methyl-6-tert-butylphenol), 4,4′-thiobis(3-methyl-6-tert-butylphenol), hydroquinone monomethyl ether, 2-hydroxybenzophenone, phenylsalicylic acid, 1,3 ,5-trimethyl-2,4,6-tris(3,5-di-tert-butyl-4-hydroxybenzyl) benzene and the like.

Thus, a need exists for improved monomer cyanoacrylate adhesive compositions, for both industrial and medical uses, having a longer shelf-life without sacrificing the performance of the adhesive.

### SUMMARY OF THE INVENTION

The present invention provides an improved monomeric adhesive composition, wherein the adhesive composition comprise a selected dual-function stabilizer. In particular embodiments, the present invention provides a monomer-containing adhesive composition comprising a polymerizable 1,1-disubstituted ethylene monomer, such as α-cyanoacrylate monomer, and at least one selected dual function stabilizer. The dual function stabilizer provides, for example, stabilization effects to the composition, and wound healing effects when utilized for medical purposes.

The combination of a polymerizable monomer with at least one selected dual function stabilizer according to the present invention provides an adhesive monomer composition with an enhanced and extended shelf-life and enhanced wound healing properties as compared to similar compositions lacking such additives. The present invention also provides an adhesive monomer composition with an enhanced and improved ability to withstand sterilization processing, such as gamma or electron beam irradiation processing, as compared to similar compositions lacking such additives. As used herein “extended shelf-life” refers to a shelf-life of at least 12 months, preferably at least 18 months, and even more preferably at least 30 months. Moreover, “enhanced wound healing capabilities” as used herein refers to the well known ability of various additives to increase the rate at which a wound heals, or to minimize the nature or effects of a wound. For example, where the dual function stabilizer is also an antioxidant, the antioxidant reduces free radicals that may otherwise hinder the ability of a wound to heal. Although it is known to add polymerization inhibitors (stabilizers) to monomeric adhesive compositions, the superiority of the use of a dual function stabilizer according to the present invention, to provide added stability and/or to enhance wound healing, has not been previously recognized.

The present invention also includes a process for enhancing the wound healing properties of such adhesive compositions, and for increasing the shelf-life of such adhesive compositions. The enhancement of the wound healing properties of the compositions includes combining a polymerizable monomer with a selected dual function stabilizer, either by itself or in combination with additional medicaments or other additives.

### DETAILED DESCRIPTION OF EMBODIMENTS

According to the present invention, a stable monomeric adhesive composition is manufactured by combining at least one selected dual function stabilizer with a composition comprising a monomer adhesive. The at least one selected dual function stabilizer according to the present invention 1) may inhibit polymerization of the monomer of the composition to a greater extent than can be achieved in prior art compositions, particularly during sterilization processing, and/or 2) may enhance the wound healing properties of the adhesive when used in medical applications.

The dual function stabilizer according to the present invention can be selected from among a range of materials that provide the desired stabilization and wound healing properties. For example, the dual function stabilizer can be selected from herbal extracts, alpha- and beta-C₁-C₃₀ hydroxycarboxylic acids, ceramides, anti-inflammatories, vasoconstrictors, and mixtures thereof.

According to embodiments, the dual function stabilizer can be a selected herbal extract. Herbal extracts particularly suitable for the present invention are antioxidants or free-radical inhibitors. As used herein, an “antioxidant” means a compound that prevents degradation caused by oxidation. Preferably, the herbal extract is oil soluble rather than water soluble, although suitable results may be achieved using herbal extracts that are both oil- and water-soluble, as well as with herbal extracts that are not oil soluble.

Examples of suitable herbal extracts that are oil soluble include, but are not limited to, chamomile, carrot root, echinacea purpurea, fennel, ginseng, grape seed, grape skin, grapefruit, guggalipids, harpogophytum, jasmine, marjoram, myrrh gum resin, onion, pine bark, red clover flower, resveratrol, rosemary, sesame, St. Johns wort, thyme, Uva Ursi (bearberry), borage seed oil, wild borage seed oil, hesperedin, quercetin, kaempherol, genistein, coumestrol, estriol, phytosterols, limonene, ethoxyquin, hydroquinone, ubiquinone (coenzyme Q), lipoic acid, N-acetyl cysteine, curcumin, derivatives thereof, mixtures thereof, and the like. Examples of suitable herbal extracts that are oil soluble and water soluble include, but are not limited to, basil leaf, bell pepper, dandelion root, date palm fruit, licorice, tomato, myricetin, derivatives thereof, mixtures thereof, and the like. Examples of suitable herbal extracts that are water soluble include, but are not limited to, black tea extracts, blackberry, black currant fruit, coffee seed, gingko leaf, green tea polyphenyls (such as epicatechin gallate and epigallocaatechin 3-O-gallate), hawthorn berries, sage, strawberry, sweet pea, vanilla fruit, neohesperidin, rutin, morin, chlorogenic acid, glutathione, derivatives thereof, mixtures thereof, and the like.

Herbal extracts particularly effective for sebum/oil control include dill, horseradish, oats, neem, beet, broccoli, tea, pumpkin, soybean, barley, walnut, flax, ginseng, poppy, avocado, pea, sesame, dandelion, wheat, nettle, cashew, pineapple, apple, asparagus, Brazilnut, chickpea, grapefruit, orange, cucumber, buckwheat, strawberry, ginko, tomato, blueberry, cowpea and grape extracts. Other herbal extracts also suitable are those of ivy horse chestnut, centella asiatica, rosmarinic acid, glycyrrizinate derivatives, alpha bisabolol, azulene and derivatives thereof, asiaticoside, sericoside, ruscogenin, escin, escolin, betulinic acid and derivatives thereof, and catechin and derivatives thereof.

In embodiments, a preferred dual function stabilizer is curcumin, or derivatives thereof such as curcuminoids. Curcumin, also referred to as tumeric yellow, difeurloylmethane, or C. I. Natural Yellow 3, provides the dual benefits of being a stabilizing agent to the adhesive composition prior to its application, as well as providing antimicrobial effects during and after application. In addition, curcumin compounds provide a characteristic color to the composition, which can assist in identifying where the adhesive composition has been applied, to help ensure proper placement and coverage. Suitable curcumin and curcumin derivatives include, but are not limited to, curcumin, demethoxycurcumin, bisdemethoxycurcumin, tetrahydrocurcumin, tetrahydrodemethoxycurcumin, tetrahydrobisdemethoxycurcumin, other curcumin metabolites, (4-hydroxy-3-methoxycinnamoyl)methane, bis(4-hydroxycinnamoyl)methane, hexahydrocurcumin, octahydrocurcumin, mixtures thereof, and the like.

According to other embodiments of the present invention, the dual function stabilizer can be a selected alpha- or beta-hydroxycarboxylic acid, such as alpha- and beta-hydroxycarboxylic acids ranging from C₂-C₃₀. The beta-hydroxycarboxylic acids are primarily exemplified by salicylic acid and C₁-C₃₀ ester and salt derivatives. Examples of suitable alpha-hydroxycarboxylic acids include but are not limited to: alpha hydroxy acetic acid (glycolic acid), alpha hydroxybenzeneacetic acid (mandelic acid), alpha hydroxypropionic acid (lactic acid), alpha hydroxybutanoic acid, alpha hydroxyhexanoic acid, alpha hydroxyoctanoic acid (alpha hydroxycaprylic acid), alpha hydroxynonanoic acid, alpha hydroxydecanoic acid, alpha hydroxyundecanoic acid, alpha hydroxydodecanoic acid (alpha hydroxylauric acid), alpha hydroxytetradecanoic acid, alpha hydrocyhexadecanoic acid, alpha hydroxyoctadecanoic acid, alpha hydroxyoctaeicosanoic acid, dicarboxylic alpha hydroxy acids, dihydroxybutanedioic acid (tartaric acid), 2-hydroxybutanedioic acid (malic acid), 2-hydroxy propanedioic acid, 2-hydroxy hexanedioic acid, 2-hydroxy octanedioic acid, 2-hydroxy decanedioic acid, 2-hydroxy dodecanedioic acid, 2-hydroxy myristicdioic acid, 2-hydroxy palmiticdioic acid, tricarboxylic alpha hydroxy acid, 2-hydroxy-1,2,3,-propanetricarboxylic acid (citric acid), 1-hydroxy-1,2,3-propanetricarboxylic acid (isocitric acid) and mixtures thereof.

C₁-C₃₀ esters and salts of alpha- and beta-hydroxycarboxylic acids (e.g. potassium, sodium, ammonium, triethanolammonium and the like salts) are also meant to be included within the term “alpha- and beta-hydroxycarboxylic acid.” Depending on the pH of the composition, a mixture of the salt and the acid may be present.

According to the present invention, preferred alpha hydroxycarboxylic acids are monocarboxylic acids, in order to improve skin penetration and efficacy. Even more preferably, the hydroxy acid is in embodiments chosen from lactic acid, glycolic acid, mandelic acid, and mixtures thereof to optimize the efficacy of compositions by increasing percutaneous absorption. Most preferred is the L-form of an alpha hydroxycarboxylic acid.

Ceramides useful for the present invention are sphingolipids or phytosphingolipids including Ceramide 1, Ceramide 3and Ceramide 6.

Anti-inflammatories of the present invention are illustrated by corticoids such as beta-methasone 17-acetate, indomethacin, ketoprofen, flufenamic acid, ibuprofen, diclofenace, diflunisal, fenclofenac, naproxen, piroxidam and sulindac. Vasoconstrictors are illustrated by compounds such as papaverine, yohimbine, visnadin, khellin, bebellin and nicotinate derivatives. Other substances within one or more of the above categories of actives include resorcinol, azelaic acid, oxamic acid and cyoctol.

The amount of dual function stabilizer that is added to the monomer composition depends on the monomer to be stabilized, the stabilizer being selected, and/or the packaging material to be used for the composition. Preferably, the dual function stabilizers of the present invention may be included in an amount of from about 0.00001 to about 40%, preferably from about 0.01 to about 20%, optimally from about 0.1 to about 10% and in some instances from about 1 to about 8% by weight of the composition. Of course, herbal extracts are usually employed at much lower levels than for instance the hydroxycarboxylic acids. Thus, the amount of herbal extracts may range from about 0.0001 to about 1%, preferably from about 0.001 to about 0.5% by weight. Preferably the amount of the hydroxycarboxylic acid component, when present in the composition according to the invention, is from about 0.5% to about 20%, more preferably from about 1% to about 15%, and most preferably from about 3.0% to about 12.0% by weight of the composition.

However, depending upon the desired stabilization and wound healing effects, lower amounts of the dual function stabilizer can be used. Thus, for example, the dual function stabilizer can be added in an amount of from about 0.01 to about 10 percent or more, preferably up to about 5 percent or more or 2.0 percent or more by weight based on the monomer. Contents outside of these ranges can be used, in embodiments, as desired. Thus, for example, where the antioxidants stabilizer is a higher molecular weight compound, greater amounts of the antioxidant can be used to obtain the desired result.

Preferably, according to the present invention, the dual function stabilizer is included in the composition in an amount not only to provide effective stabilization of the monomeric adhesive composition, but also to provide wound healing properties to the composition. Thus, for example, the dual function stabilizer is included in an amount that provides sufficient residual antioxidant stabilizer in a formed polymeric material, such that the antioxidant can diffuse out of the formed polymer over time. By such diffusion, the stabilizer is made available to an adjacent tissue and/or a surrounding environment to impart wound healing properties.

As used herein an amount of dual function stabilizer sufficient or effective “to stabilize” the monomer composition refers to an amount of stabilizer sufficient to prevent the viscosity of a sterilized monomer composition from increasing to more than 200%, and preferably 150%, of the composition's initial viscosity after sterilization. Suitable monomer composition stability, in terms of composition viscosity, according to the present invention is disclosed in U.S. patent application Ser. No. 09/374,207 filed Aug. 12, 1999, the entire disclosure of which is incorporated herein by reference.

An indication of premature polymerization in 1,1-disubstituted ethylene monomer compositions, such as α-cyanoacrylate monomer compositions in particular, is an increase in viscosity of the composition over time. That is, as the composition polymerizes, the viscosity of the composition increases. If the viscosity becomes too high, i.e., too much premature polymerization has occurred, the composition becomes unsuitable for its intended use or becomes very difficult to apply. Thus, while some polymerization or thickening of the composition may occur, such as can be measured by changes in viscosity of the composition, such change is not so extensive as to destroy or significantly impair the usefulness of the composition. However, the present invention, by providing a dual function stabilizer in the composition stored in the container, decreases or prevents the premature polymerization of the composition, and thereby provides better control over the viscosity of the composition.

In embodiments of the present invention, the dual function stabilizer is included in the composition in an amount effective to provide effective stabilization of the composition. Thus, for example, the dual function stabilizer is contained in an amount effective to stabilize the composition over a desired shelf-life of the product, and/or to stabilize the composition during and after any applicable sterilization procedure. A particular advantage of the stabilizers is that they provide effective stabilization of the composition during sterilization procedures, such as irradiation, dry heat, and/or chemical sterilization processes. Accordingly, and as necessary, the amount of the dual function stabilizer added to the composition can be varied depending upon, for example, the projected shelf-life of the composition and/or the kind and degree of selected sterilization processing. Such selection and variation of the stabilizer can be performed by one of ordinary skill in the art with only routine experimentation.

In embodiments of the present invention, particularly where the composition is to be used as a wound dressing or is otherwise being applied to tissue, the dual function stabilizer can be included in the composition for its wound healing properties as well as for its stabilization properties. In these embodiments, if desired, the amount of the stabilizer contained in the composition can be increased, so that an effective amount of the stabilizer remains present in the resultant polymer. When so present, the dual function stabilizer is generally released or diffuses from the polymer, either immediately or over time, into the tissue. Accordingly, one or more of the above-described additives can be included in the composition for their wound-healing effects.

In addition, according to the present invention, the above-described dual function stabilizers can, additionally or alternatively, be included in a container that is used to contain polymerizable monomer adhesive compositions, such as 1,1-disubstituted adhesive compositions and cyanoacrylate adhesive compositions. The benefits of such inclusion, as well as a description of methods of including materials in the container itself, are disclosed in U.S. patent application Ser. No. 09/657,913, filed Sep. 8, 2000, the entire disclosure of which is incorporated herein by reference. The methods disclosed therein for antioxidants and other stabilizers are also equally applicable to the dual function stabilizers of the present invention. Similar disclosure is also included in U.S. patent application Ser. No. 09/430,289, filed Oct. 29, 1999, the entire disclosure of which is incorporated herein by reference.

According to the present invention, the container can be any suitable container used to contain a polymerizable monomeric adhesive composition. Thus, the container can be formed out of any suitable material, and in any shape, size and/or construction, as desired. Suitable container constructions are disclosed, for example, in the above-referenced co-pending U.S. patent application Ser. Nos. 09/430,289 and 09/657,913, the entire disclosure of which is incorporated herein by reference.

In embodiments, the method of the present invention further comprises sterilizing the 1,1-disubstituted ethylene monomer composition and/or its packaging, either prior to, or subsequent to, dispensing into the container. Suitable sterilization methods according to the present invention are disclosed in U.S. patent application Ser. No. 09/374,207 filed Aug. 12, 1999, the entire disclosure of which is incorporated herein by reference.

Sterilization of the monomer composition and/or its packaging can be accomplished by techniques known to one of ordinary skill in the art, and is preferably accomplished by methods including, but not limited to, chemical, physical, and/or irradiation methods. Examples of chemical methods include, but are not limited to, exposure to ethylene oxide or hydrogen peroxide vapor. Examples of physical methods include, but are not limited to, sterilization by heat (dry or moist) or retort canning. Examples of irradiation methods include, but are not limited to, gamma irradiation, electron beam irradiation, and microwave irradiation. A preferred method is electron beam irradiation, as described in U.S. Pat. No. 6,143,805, the entire disclosure of which is incorporated herein by reference, as well as gamma irradiation. The composition must show low levels of toxicity to living tissue during its useful life. In preferred embodiments of the present invention, the composition is sterilized to provide a Sterility Assurance Level (SAL) of at least 10⁻³. In embodiments, the Sterility Assurance Level may be at least 10⁻⁴, or may be at least 10⁻⁵, or may be at least 10⁻⁶.

According to the invention, the combination of at least one dual function stabilizer provides sufficient inhibition of polymerization of the monomer (i.e., stabilization of the composition) that sterility can be achieved without the unacceptable levels of polymerization or increases in cure rate due to over-stabilization that result from methods disclosed in the prior art. For example, sterilized compositions according to embodiments of the present invention show an increase in viscosity of no more than 300%, and preferably less than 150%, as a result of sterilization. Viscosity levels can be determined by known techniques. For example, viscosity can be determined at room temperature (approximately 21-25° C.) using a Brookfield Cone-Plate Viscometer. The instrument is standardized using a Viscosity Reference Standard in the same range as the sample to be tested. Each sample is measured three times, and an average value determined and recorded.

Preferred monomer compositions of the present invention, and polymers formed therefrom, are useful as tissue adhesives, sealants for preventing bleeding or for covering open wounds, and in other absorbable and non-absorbable biomedical applications. They find uses in, for example, apposing surgically incised or traumatically lacerated tissues; retarding blood flow from wounds; dressing burns; dressing skin or other superficial or surface wounds (such as abrasions, chaffed or raw skin, ulceration and/or stomatitis); hernia repair; meniscus repair; and aiding repair and re-growth of living tissue. Compositions of the present invention, and polymers formed therefrom, are also useful in industrial and home applications, for example in bonding rubbers, plastics, wood, composites, fabrics, and other natural and synthetic materials.

The monomer (including prepolymeric) adhesive composition may include one or more polymerizable monomers. Preferred monomers that may be used in this invention are readily polymerizable, e.g. anionically polymerizable or free radical polymerizable, or polymerizable by zwitterions or ion pairs to form polymers. Such monomers include those that form polymers, that may, but do not need to, biodegrade. Such monomers are disclosed in, for example, U.S. Pat. Nos. 5,328,687 and 5,928,611 to Leung et al., U.S. patent application Ser. No. 09/430,177, filed on Oct. 29, 1999, and U.S. Pat. No. 6,183,593, which are hereby incorporated in their entirety by reference herein. Preferred monomers include 1,1-disubstituted ethylene monomers, such as α-cyanoacrylates including, but not limited to, alkyl α-cyanoacrylates having an alkyl chain length of from about 1 to about 20 carbon atoms or more, preferably from about 3 to about 8 carbon atoms.

The α-cyanoacrylates of the present invention can be prepared according to several methods known in the art. U.S. Pat. Nos. 2,721,858, 3,254,111, 3,995,641, and 4,364,876, each of which is hereby incorporated in its entirety by reference herein, disclose methods for preparing α-cyanoacrylates.

Preferred α-cyanoacrylate monomers used in this invention include methyl cyanoacrylate, ethyl cyanoacrylate, n-butyl cyanoacrylate, 2-octyl cyanoacrylate, methoxyethyl cyanoacrylate, ethoxyethyl cyanoacrylate, dodecyl cyanoacrylate, 2-ethylhexyl cyanoacrylate, butyl cyanoacrylate, 3-methoxybutyl cyanoacrylate, 2-butoxyethyl cyanoacrylate, 2-isopropoxyethyl cyanoacrylate, 1-methoxy-2-propyl cyanoacrylate, hexyl cyanoacrylate, or dodecylcyanoacrylate.

Suitable cyanoacrylates for use in the present invention also include, but are not limited to, alkyl ester cyanoacrylate monomers such as those having the formula

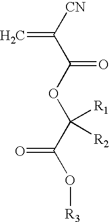

wherein R₁ and R₂ are, independently H, a straight, branched or cyclic alkyl, or are combined together in a cyclic alkyl group, and R₃ is a straight, branched or cyclic alkyl group. Preferably, R₁is H or a C₁, C₂ or C₃ alkyl group, such as methyl or ethyl; R₂ is H or a C₁, C₂ or C₃ alkyl group, such as methyl or ethyl; and R₃ is a C₁-C₁₆ alkyl group, more preferably a C₁-C₁₀ alkyl group, such as methyl, ethyl, propyl, butyl, pentyl, hexyl, heptyl, octyl, nonyl or decyl, and even more preferably a C₂, C₃ or C₄ alkyl group. Such alkyl ester cyanoacrylates and other suitable monomers are disclosed in, for example, U.S. patent application Ser. Nos. 09/630,437, filed Aug. 2, 2000, and 09/919,877, filed Aug. 2, 2001, the entire disclosures of which are incorporated herein by reference.

Examples of preferred alkyl ester cyanoacrylates include, but are not limited to, butyl lactoyl cyanoacrylate (BLCA), butyl glycoloyl cyanoacrylate (BGCA), ethyl lactoyl cyanoacrylate (ELCA), and ethyl glycoloyl cyanoacrylate (EGCA). BLCA may be represented by the above formula, wherein R₁ is H, R₂ is methyl and R₃ is butyl. BGCA may be represented by the above formula, wherein R₁ is H, R₂ is H and R₃ is butyl. ELCA may be represented by the above formula, wherein R₁ is H, R₂ is methyl and R₃ is ethyl. EGCA may be represented by the above formula, wherein R₁ is H, R₂ is H and R₃ is ethyl.

The composition may optionally also include at least one other plasticizing agent that assists in imparting flexibility to the polymer formed from the monomer. The plasticizing agent preferably contains little or no moisture and should not significantly affect the stability or polymerization of the monomer. Examples of suitable plasticizers include but are not limited to isopropyl myristate, isopropyl palimtate, tributyl citrate, acetyl tri-n-butyl citrate (ATBC), polymethylmethacrylate, polydimethylsiloxane, polyester glutarates; polyester adipates; polyester sebacates; and others as listed in U.S. Pat. No. 6,183,593, the disclosure of which is incorporated in its entirety by reference herein.

The composition may also optionally include at least one thixotropic agent. Suitable thixotropic agents are known to the skilled artisan and include, but are not limited to, silica gels such as those treated with a silyl isocyanate, and optionally surface treated titanium dioxide. Examples of suitable thixotropic agents and thickeners are disclosed in, for example, U.S. Pat. No. 4,720,513, and U.S. patent application Ser. No. 09/374,207 filed Aug. 12, 1999, the disclosures of which are hereby incorporated in their entireties by reference herein.

The composition may optionally also include thickeners. Suitable thickeners may include poly (2-ethylhexy methacrylate), poly(2-ethylhexyl acrylate) and others as listed in U.S. patent application Ser. No. 09/472,392 filed Dec. 23, 1999, the disclosure of which is incorporated by reference herein in its entirety.

The composition may also optionally include at least one natural or synthetic rubber to impart impact resistance. Suitable rubbers are known to the skilled artisan. Such rubbers include, but are not limited to, dienes, styrenes, acrylonitriles, and mixtures thereof. Examples of suitable rubbers are disclosed in, for example, U.S. Pat. Nos. 4,313,865 and 4,560,723, the disclosures of which are hereby incorporated in their entireties by reference herein.

The composition may optionally also include one or more additional stabilizers, such as both at least one anionic vapor phase stabilizer and at least one anionic liquid phase stabilizer. These stabilizing agents may further inhibit premature polymerization. Suitable stabilizers may include those listed in U.S. Pat. No. 6,183,593, the disclosure of which is incorporated by reference herein in its entirety. Suitable stabilizing agents also include any of the known anionic and free radical stabilizing agents.

Where an one or more additional stabilizers is included in the compositions, a preferred additional stabilizer in embodiments is an antioxidant stabilizer, such as those disclosed in U.S. patent application Ser. No. 09/657,913, filed Sep. 8, 2000, the disclosure of which is incorporated by reference herein in its entirety.

Suitable antioxidant stabilizer can be selected from among known antioxidants, including, but not limited to, vitamin E (C₂₉H₅₀O₂) (including alpha-tocopherol (C₂₉H₅₀O₂), beta-tocopherol (C₂₈H₄₈O₂), gamma-tocopherol (C₂₈H₄₈O₂) and delta-tocopherol (C₂₇H₄₆O₂) and derivatives thereof, vitamin K (including phylloquinone (C₃₁H₄₆O₂), menaquinone (e.g. menaquinone 4 (C₃₁H₄₀ O₂)), and menadione (C₁₁H₈O₂) and derivatives thereof, including but not limited to vitamin K₁, chromanol and vitamin K₁ chromenol, vitamin C (ascorbic acid (C₆H₈O₆)) and derivatives thereof, pentamethyl chromanol (C₁₄H₂₀O₂), non-phenolic antioxidants, octyl gallate (C₁₄H₂₀O₅) and pentamethyl benzofuranol (C₁₃H₁₈O₂). A preferred vitamin E antioxidant is any of the series of IRGANOX® brand vitamin E (available from Ciba Specialty Chemical Co.). A preferred pentamethyl chromanol is 2,2,5,7,8-pentamethyl-6-chromanol. Derivatives of the described compounds, particularly where the moieties are benzopyranols or benzofuranols, are also suitable.

Other suitable antioxidant stabilizers include compounds of the following formula:

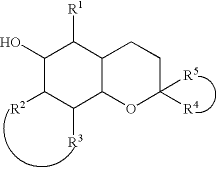

where R¹, R², R³, R⁴, and R⁵ are independently selected from hydrogen, substituted alkyl or unsubstituted alkyl groups having from, for example, 1 to about 20 carbon atoms. Furthermore, suitable compounds include those of the above formula where R² and R³, and/or R⁴ and R⁵, may optionally form cyclic groups having from about 2 to about 40 carbon atoms, preferably from about 2 or about 3 to about 6 or about 8 carbon atoms.

The compositions may also include pH modifiers to control the rate of degradation of the resulting polymer, as disclosed in U.S. patent application Ser. No. 08/714,288, filed Sep. 18, 1996, the entire disclosure of which is hereby incorporated by reference herein in its entirety.

Compositions of the present invention may also include at least one biocompatible agent effective to reduce active formaldehyde concentration levels produced during in vivo biodegradation of the polymer (also referred to herein as “formaldehyde concentration reducing agents”). Preferably, this component is a formaldehyde scavenger compound. Examples of formaldehyde scavenger compounds useful in this invention include sulfites; bisulfites; mixtures of sulfites and bisulfites, etc. Additional examples of formaldehyde scavenger compounds useful in this invention and methods for their implementation can be found in U.S. Pat. Nos. 5,328,687, 5,514,371, 5,514,372, 5,575,997, 5,582,834 and 5,624,669, all to Leung et al., which are hereby incorporated herein by reference in their entireties.

To improve the cohesive strength of adhesives formed from the compositions of this invention, difunctional monomeric cross-linking agents may be added to the monomer compositions of this invention. Such crosslinking agents are known. U.S. Pat. No. 3,940,362 to Overhults, which is hereby incorporated herein in its entirety by reference, discloses exemplary cross-linking agents.

The compositions of this invention may further contain a fibrous reinforcement and colorants such as dyes, pigments, and pigment dyes. Examples of suitable fibrous reinforcements include PGA microfibrils, collagen microfibrils, and others as described in U.S. Pat. No. 6,183,593, the disclosure of which is incorporated by reference herein in its entirety.

The polymerizable compositions useful in the present invention may also further contain one or more medicaments, preferably one or more non-antioxidant medicaments. Suitable medicaments include, but are not limited to, antibiotics, antimicrobials, antiseptics, bacteriocins, bacteriostats, disinfectants, steroids, anesthetics, antifungal agents, anti-inflammatory agents (other than the dual function stabilizers of the present invention), antibacterial agents, antiviral agents, antitumor agents, growth promoting substances, antioxidants (other than the dual function stabilizers of the present invention), or mixtures thereof. Suitable specific medicaments are disclosed in, for example, U.S. patent application Ser. No. 09/430,177, filed Oct. 29, 1999, the entire disclosure of which is incorporated herein by reference.

The polymerizable compositions useful in the present invention may also further contain one or more preservatives. The preservatives may be present, for example, for prolonging the storage life of the composition and/or for destroying and/or usefully suppressing the growth or metabolism of a variety of microscopic or submicroscopic life forms, either in the composition itself, or in or on a substrate to which the composition may be applied. Suitable preservatives, and methods for selecting them and incorporating them into adhesive compositions, are disclosed in U.S. patent application Ser. No. 09/430,180, the entire disclosure of which is incorporated herein by reference.

In embodiments of the present invention, the composition and/or its applicator may contain materials such as a polymerization initiator, accelerator, rate-modifier, and/or cross-linking agent for initiating polymerization and/or cross-linking of the polymerizable monomer material. Suitable materials and applicators and packaging systems are disclosed in U.S. Pat. No. 5,928,611 and U.S. patent application Ser. Nos. 09/430,177, 09/430,176, 09/430,289, 09/430,290, and 09/430,180 filed Oct. 29, 1999; 09/343,914 filed Jun. 30, 1999; 09/385,030 filed Aug. 30, 1999; and 09/176,889 filed Oct. 22, 1998; the entire disclosures of which are incorporated herein by reference.

### EXAMPLES

### Example 1

A 2-octyl Cyanoacrylate monomer (2-OCA) composition is prepared by the addition of 0.5 wt. percent of curcumin. The monomer composition is stirred until all the curcumin is dissolved. The curcumin dissolves in the 2-OCA to provide a medically acceptable monomer composition. The formulation is suitable for evaluated of its wound healing property.

### Example 2

Pigs are anesthetized and the entire dorsal, thoracic region is shaved using an electric clipper. Portions of the area are shaved using a single edge razor and soap as a lubricant. Care is taken to avoid abrasions during shaving. The shaved surface is antiseptically prepared using a non-antimicrobial soap solution followed by 3 rinses with water and a final rinse with 70% isopropyl alcohol. Wounds measuring approximately 0.7 cm×0.7 cm are made on the skin with the aid of a Dermatome. Wounds are made to a depth of approximately 300 μm.

Formulations are randomly applied to the wounds. The formulations include a control 2-OCA formulation (similar to the formulation of Example 1 but without added curcumin); the 2-OCA formulation of Example 1; TEGADERM®; and no formulation (i.e., the wound is exposed to air).

After a 4-day exposure period, animals are anesthetized under 3% isoflurane and five wounds per treatment group are excised using a dermatome set to cut 700 μm deep. The harvested wounds are subjected to 0.5mol/L NaBr for 24 hours at 37 ±2° C. in 6-well plates. After incubation, the dermis and epidermis are separated using fine forceps, and the epidermal integrity is determined by macroscopic examination of the layer for interruptions in the continuum of the epidermal sheet. This procedure is repeated on days 5-8 after the initial injury. The excision sites are dressed with the test article to stop the bleeding and seal the wound. Animals are euthanized with a veterinary barbiturate after collection of test and control sites on day 8.

The table below shows the results of the testing, showing the percent of the wound that is completely re-epithelialized (i.e., healed).

| Days Post- | 2-OCA | 2-OCA w/ | | Air |
|---|---|---|---|---|
| Treatment | control | curcumin | TEGADERM ® | exposed |
| 4 | 0 | 60 | 0 | 0 |
| 5 | 20 | 100 | 20 | 0 |
| 6 | 80 | 100 | 80 | 0 |
| 7 | 80 | 100 | 100 | 100 |
| 8 | 80 | 100 | 80 | 80 |

### Example 3

A 2-octyl Cyanoacrylate monomer (2-OCA) composition is prepared by the addition of 3000 ppm tetrahydrocurcumin (THC). The monomer composition is stirred until all the THC dissolves. A 2-OCA formulation without added THC is used as a control. Fluorinated high-density polyethylene bottles are filled with the formulations and subsequently exposed to electron beam sterilization at 15 KGy. The sterilized bottles are maintained at elevated temperature for 12 days. The stability of the formulations are evaluated at 6 and 12 days post sterilization for changes in viscosity. The results are shown in the following Table.

| | Change | change | change |
|---|---|---|---|
| | in viscosity | in viscosity | in viscosity |
| Formulation | post e-beam | after 6 days | after 12 days |
| 2-OCA + 3000 ppm | 1.3 cPs | 4.7 cPs | 57.8 cPs |
| THC | | | |
| 2-OCA control | 1.1 cPs | 5.2 cPs | 56.2 cPs |

### Example 4

A 2-octyl Cyanoacrylate monomer (2-OCA) composition is prepared by the addition of 2500 ppm of tetrahydrocurcumin (THC) and 2500 ppm of 2,2,5,7,8-pentamethyl-6-chromanol. The monomer composition is stirred until both the THC and chromanol dissolve. A 2-OCA formulation without added THC or chromanol is used as a control. Fluorinated high-density polyethylene bottles are filled with the formulations and subsequently exposed to electron beam sterilization at 15 KGy. The bottles are maintained at elevated temperature for 12 days. The stability of the formulations are evaluated at 6 and 12 days post sterilization for changes in viscosity. The results are shown in the following table.

| | Change in | Change in | Change in |
|---|---|---|---|
| | viscosity | viscosity | viscosity after |
| Formulation | Post ebeam | after 6 days | 12 days |
| 2-OCA + 2500 ppm THC + | 0.3 cPs | 3.7 cPs | 34.8 cPs |
| 2500 ppm of chromanol | | | |
| 2-OCA + 5000 ppm | 0.7 cPs | 4.2 cPs | 45.5 cPs |
| of chromanol | | | |
| 2-OCA | 1.6 cPs | 7.8 cPs | 81 cPs |

While the invention has been described with reference to preferred embodiments, the invention is not limited to the specific examples given, and other embodiments and modifications can be made by those skilled in the art without departing from the spirit and the scope of the invention.

What is claimed is:

1. An adhesive composition comprising:
a polymerizable 1,1-disubstituted ethylene monomer; and
at least one stabilizer selected from the group consisting of herbal extracts, alpha- and beta- hydroxycarboxylic acids and salts thereof, ceramides, anti-inflammatories, vasoconstrictors, and mixtures thereof.

2. The composition of claim 1, wherein said stabilizer is an herbal extract.

3. The composition of claim 2, wherein said herbal extract is an oil-soluble herbal extract.

4. The composition of claim 3, wherein said herbal extract is selected from the group consisting of chamomile, carrot root, echinacea purpurea, fennel, ginseng, grape seed, grape skin, grapefruit, guggalipids, harpogophytum, jasmine, marjoram, myrrh gum resin, onion, pine bark, red clover flower, resveratrol, rosemary, sesame, St. Johns wort, thyme, Uva Ursi (bearberry), borage seed oil, wild borage seed oil, hesperedin, quercetin, kaempherol, genistein, coumestrol, estriol, phytosterols, limonene, ethoxyquin, hydroquinone, ubiquinone (coenzyme Q), lipoic acid, N-acetyl cysteine, curcumin, basil leaf, bell pepper, dandelion root, date palm fruit, licorice, tomato, myricetin, derivatives thereof, and mixtures thereof.

5. The composition of claim 3, wherein said herbal extract is selected from the group consisting of chamomile, carrot root, echinacea purpurea, fennel, ginseng, grape seed, grape skin, grapefruit, guggalipids, harpogophytum, jasmine, marjoram, myrrh gum resin, onion, pine bark, red clover flower, resveratrol, rosemary, sesame, St. Johns wort, thyme, Uva Ursi (bearberry), borage seed oil, wild borage seed oil, hesperedin, quercetin, kaempherol, genistein, coumestrol, estriol, phytosterols, limonene, ethoxyquin, hydroquinone, ubiquinone (coenzyme Q), lipoic acid, N-acetyl cysteine, curcumin, derivatives thereof, and mixtures thereof.

6. The composition of claim 3, wherein said herbal extract is selected from the group consisting of dill, horseradish, oats, neem, beet, broccoli, tea, pumpkin, soybean, barley, walnut, flax, ginseng, poppy, avocado, pea, sesame, dandelion, wheat, nettle, cashew, pineapple, apple, asparagus, Brazilnut, chickpea, grapefruit, orange, cucumber, buckwheat, strawberry, ginko, tomato, blueberry, cowpea, grape extracts, ivy horse chestnut, centella asiatica, rosmarinic acid, glycyrrizinate derivatives, alpha bisabolol, azulene, asiaticoside, sericoside, ruscogenin, escin, escolin, betulinic acid, catechin, derivatives thereof, and mixtures thereof.

7. The composition of claim 1, wherein said stabilizer is curcumin or a curcumin derivative.

8. The composition of claim 7, wherein said stabilizer is selected from the group consisting of curcumin, demethoxycurcumin, bisdemethoxycurcumin, tetrahydrocurcumin, tetrahydrodemethoxycurcumin, tetrahydrobisdemethoxycurcumin, other curcumin metabolites, (4-hydroxy-3-methoxycinnamoyl)methane, bis(4-hydroxycinnamoyl)methane, hexahydrocurcumin, octahydrocurcumin, and mixtures thereof.

9. The composition of claim 1, wherein said stabilizer is an alpha- or beta-C₁-C₃₀ hydroxycarboxylic acid or salt thereof.

10. The composition of claim 9, wherein said stabilizer is selected from the group consisting of alpha- and beta-C₁-C₃₀ hydroxycarboxylic acids and salts thereof.

11. The composition of claim 9, wherein said stabilizer is selected from the group consisting of salicylic acid, alpha hydroxy acetic acid, alpha hydroxybenzeneacetic acid, alpha hydroxypropionic acid, alpha hydroxybutanoic acid, alpha hydroxyhexanoic acid, alpha hydroxyoctanoic acid, alpha hydroxynonanoic acid, alpha hydroxydecanoic acid, alpha hydroxyundecanoic acid, alpha hydroxydodecanoic acid, alpha hydroxytetradecanoic acid, alpha hydrocyhexadecanoic acid, alpha hydroxyoctadecanoic acid, alpha hydroxyoctaeicosanoic acid, dicarboxylic alpha hydroxy acids, dihydroxybutanedioic acid (tartaric acid), 2-hydroxybutanedioic acid, 2-hydroxy propanedioic acid, 2-hydroxy hexanedioic acid, 2-hydroxy octanedioic acid, 2-hydroxy decanedioic acid, 2-hydroxy dodecanedioic acid, 2-hydroxy myristicdioic acid, 2-hydroxy palmiticdioic acid, tricarboxylic alpha hydroxy acid, 2-hydroxy-1,2,3,-propanetricarboxylic acid, 1-hydroxy-1,2,3-propanetricarboxylic acid, and mixtures thereof.

12. The composition of claim 1, wherein said stabilizer is a ceramide.

13. The composition of claim 12, wherein said ceramide is selected from the group consisting of Ceramide 1, Ceramide 3, Ceramide 6, and mixtures thereof.

14. The composition of claim 1, wherein said stabilizer is an anti-inflammatory.

15. The composition of claim 14, wherein said anti-inflammatory is selected from the group consisting of beta-methasone 17-acetate, indomethacin, ketoprofen, flufenamic acid, ibuprofen, diclofenace, diflunisal, fenclofenac, naproxen, piroxidam, sulindac, and mixtures thereof.

16. The composition of claim 1, wherein said stabilizer is a vasoconstrictor.

17. The composition of claim 16, wherein said vasoconstrictor is selected from the group consisting of papaverine, yohimbine, visnadin, khellin, bebellin, nicotinate derivatives, and mixtures thereof.

18. The composition of claim 1, wherein said monomer is an α-cyanoacrylate.

19. The composition of claim 1, wherein said monomer is at least one member selected from the group consisting of methyl cyanoacrylate, ethyl cyanoacrylate, n-butyl cyanoacrylate, 2-octyl cyanoacrylate, methoxyethyl cyanoacrylate, ethoxyethyl cyanoacrylate, hexyl cyanoacrylate, dodecyl cyanoacrylate, butyl lactoyl cyanoacrylate, butyl glycoloyl cyanoacrylate, ethyl lactoyl cyanoacrylate, and ethyl glycoloyl cyanoacrylate.

20. The composition of claim 1, wherein said stabilizer is present in said composition in a concentration of from about 0.01 to about 10.0% by weight.

21. The composition of claim 1, wherein said stabilizer is present in said composition in a concentration of from about 0.01 to about 5.0% by weight.

22. The composition of claim 1, wherein said composition has a Sterility Assurance Level of not better than 10⁻³.

23. The composition of claim 1, wherein said composition is sterilized to a Sterility Assurance Level of at least 10⁻³.

24. The composition of claim 1, further comprising a non-stabilizer medicament.

25. The composition of claim 1, further comprising a plasticizer.

26. The composition of claim 1, wherein said stabilizer is present in an amount sufficient to stabilize said composition during sterilization.

27. The composition of claim 1, wherein said stabilizer is present in an amount sufficient to stabilize said composition during and subsequent to sterilization.

28. The composition of claim 1, wherein said stabilizer is present in an amount effective to provide wound healing when said composition is applied to a tissue surface.

29. The composition of claim 1, wherein said composition further comprises at least one antioxidant stabilizer.

30. The composition of claim 29, wherein said at least one antioxidant stabilizer is selected from the group consisting of vitamin E and derivatives thereof, vitamin K and derivatives thereof, vitamin C, pentamethyl chromanol, non-phenolic antioxidants, octyl gallate, and pentamethylbenzofuranol.

31. The composition of claim 29, wherein said at least one antioxidant stabilizer is pentamethyl chromanol.

32. A method of making the adhesive composition of claim 1, comprising combining the polymerizable 1,1-disubstituted ethylene monomer and the stabilizer.

33. A method of making a sterile polymerizable 1,1-disubstituted monomer adhesive composition comprising:
dispensing the polymerizable 1,1-disubstituted monomer adhesive composition of claim 1 into a container;
sealing said container; and
sterilizing the composition in the container.

34. A method of making a sterile polymerizable 1,1-disubstituted monomer adhesive composition comprising:
dispensing a polymerizable 1,1-disubstituted monomer adhesive composition into a container;
sealing said container; and
sterilizing the composition in the container to a Sterility Assurance Level of at least 10⁻³,
wherein said polymerizable 1,1-disubstituted monomer composition comprises:
a polymerizable 1,1-disubstituted ethylene monomer; and
at least one stabilizer selected from the group consisting of herbal extracts, alpha- and beta-hydroxycarboxylic acids and salts thereof, ceramides, anti-inflammatories, vasoconstrictors, and mixtures thereof.

35. The method of claim 34, wherein said sterilizing is by dry heat, gamma irradiation, electron beam irradiation, or microwave irradiation.

36. The method of claim 34, wherein said sterilizing is by electron beam irradiation.

37. The method of claim 34, wherein said sterilizing is by gamma irradiation.

38. The method of claim 34, wherein the sterilized composition has a viscosity less than 150% of a viscosity of said composition prior to sterilizing.

39. The method of claim 34, wherein said stabilizer is an herbal extract.

40. The method of claim 39, wherein said herbal extract is an oil-soluble herbal extract.

41. The method of claim 40, wherein said herbal extract is selected from the group consisting of chamomile, carrot root, echinacea purpurea, fennel, ginseng, grape seed, grape skin, grapefruit, guggalipids, harpogophytum, jasmine, marjoram, myrrh gum resin, onion, pine bark, red clover flower, resveratrol, rosemary, sesame, St. Johns wort, thyme, Uva Ursi (bearberry), borage seed oil, wild borage seed oil, hesperedin, quercetin, kaempherol, genistein, coumestrol, estriol, phytosterols, limonene, ethoxyquin, hydroquinone, ubiquinone (coenzyme Q), lipoic acid, N-acetyl cysteine, curcumin, basil leaf, bell pepper, dandelion root, date palm fruit, licorice, tomato, myricetin, derivatives thereof, and mixtures thereof.

42. The method of claim 40, wherein said herbal extract is selected from the group consisting of chamomile, carrot root, echinacea purpurea, fennel, ginseng, grape seed, grape skin, grapefruit, guggalipids, harpogophytum, jasmine, marjoram, myrrh gum resin, onion, pine bark, red clover flower, resveratrol, rosemary, sesame, St. Johns wort, thyme, Uva Ursi (bearberry), borage seed oil, wild borage seed oil, hesperedin, quercetin, kaempherol, genistein, coumestrol, estriol, phytosterols, limonene, ethoxyquin, hydroquinone, ubiquinone (coenzyme Q), lipoic acid, N-acetyl cysteine, curcumin, derivatives thereof, and mixtures thereof.

43. The method of claim 40, wherein said herbal extract is selected from the group consisting of dill, horseradish, oats, neem, beet, broccoli, tea, pumpkin, soybean, barley, walnut, flax, ginseng, poppy, avocado, pea, sesame, dandelion, wheat, nettle, cashew, pineapple, apple, asparagus, Brazilnut, chickpea, grapefruit, orange, cucumber, buckwheat, strawberry, ginko, tomato, blueberry, cowpea, grape extracts, ivy horse chestnut, centella asiatica, rosmarinic acid, glycyrrizinate derivatives, alpha bisabolol, azulene, asiaticoside, sericoside, ruscogenin, escin, escolin, betulinic acid, catechin, derivatives thereof, and mixtures thereof.

44. The method of claim 34, wherein said stabilizer is curcumin or a curcumin derivative.

45. The method of claim 44, wherein said stabilizer is selected from the group consisting of curcumin, demethoxycurcumin, bisdemethoxycurcumin, tetrahydrocurcumin, tetrahydrodemethoxycurcumin, tetrahydrobisdemethoxycurcumin, other curcumin metabolites, (4-hydroxy-3-methoxycinnamoyl)methane, bis(4-hydroxycinnamoyl)methane, hexahydrocurcumin, octahydrocurcumin, and mixtures thereof.

46. The method of claim 34, wherein said stabilizer is an alpha- or beta-C₁-C₃₀ hydroxycarboxylic acid or salt thereof.

47. The method of claim 46, wherein said stabilizer is selected from the group consisting of alpha- and beta-C₁-C₃₀ hydroxycarboxylic acids and salts thereof.

48. The method of claim 46, wherein said stabilizer is selected from the group consisting of salicylic acid, alpha hydroxy acetic acid, alpha hydroxybenzeneacetic acid, alpha hydroxypropionic acid, alpha hydroxybutanoic acid, alpha hydroxyhexanoic acid, alpha hydroxyoctanoic acid, alpha hydroxynonanoic acid, alpha hydroxydecanoic acid, alpha hydroxyundecanoic acid, alpha hydroxydodecanoic acid, alpha hydroxytetradecanoic acid, alpha hydrocyhexadecanoic acid, alpha hydroxyoctadecanoic acid, alpha hydroxyoctaeicosanoic acid, dicarboxylic alpha hydroxy acids, dihydroxybutanedioic acid (tartaric acid), 2-hydroxybutanedioic acid, 2-hydroxy propanedioic acid, 2-hydroxy hexanedioic acid, 2-hydroxy octanedioic acid, 2-hydroxy decanedioic acid, 2-hydroxy dodecanedioic acid, 2-hydroxy myristicdioic acid, 2-hydroxy palmiticdioic acid, tricarboxylic alpha hydroxy acid, 2-hydroxy-1,2,3,-propanetricarboxylic acid, 1-hydroxy-1,2,3-propanetricarboxylic acid, and mixtures thereof.

49. The method of claim 34, wherein said stabilizer is a ceramide.

50. The method of claim 49, wherein said ceramide is selected from the group consisting of Ceramide 1, Ceramide 3, Ceramide 6, and mixtures thereof.

51. The method of claim 34, wherein said stabilizer is an anti-inflammatory.

52. The method of claim 51, wherein said anti-inflammatory is selected from the group consisting of beta-methasone 17-acetate, indomethacin, ketoprofen, flufenamic acid, ibuprofen, diclofenace, diflunisal, fenclofenac, naproxen, piroxidam, sulindac, and mixtures thereof.

53. The method of claim 34, wherein said stabilizer is a vasoconstrictor.

54. The method of claim 53, wherein said vasoconstrictor is selected from the group consisting of papaverine, yohimbine, visnadin, khellin, bebellin, nicotinate derivatives, and mixtures thereof.

55. The method of claim 34, wherein said monomer is an α-cyanoacrylate.

56. The method of claim 34, wherein said monomer is at least one member selected from the group consisting of methyl cyanoacrylate, ethyl cyanoacrylate, n-butyl cyanoacrylate, 2-octyl cyanoacrylate, methoxyethyl cyanoacrylate, ethoxyethyl cyanoacrylate, hexyl cyanoacrylate, dodecyl cyanoacrylate, butyl lactoyl cyanoacrylate, butyl glycoloyl cyanoacrylate, ethyl lactoyl cyanoacrylate, and ethyl glycoloyl cyanoacrylate.

57. The method of claim 34, wherein said stabilizer is present in said composition in a concentration of from about 0.01 to about 10.0% by weight.

58. The method of claim 34, wherein said stabilizer is present in said composition in a concentration of from about 0.01 to about 5.0% by weight.

59. The method of claim 34, wherein said composition has a total volume in said container of less than 10 milliliters.

60. The method of claim 34, wherein said composition further comprises at least one antioxidant stabilizer.

61. The method of claim 60, wherein said at least one antioxidant stabilizer is selected from the group consisting of vitamin E and derivatives thereof, vitamin K and derivatives thereof, vitamin C, pentamethyl chromanol, non-phenolic antioxidants, octyl gallate, and pentamethylbenzofuranol.

62. The method of claim 60, wherein said at least one antioxidant stabilizer is pentamethyl chromanol.

63. A sterile composition made by the method of claim 34.

64. A polymerized film made by polymerizing the composition of claim 1.

65. A method of treating tissue comprising:
applying the composition of claim 1 to tissue; and
allowing the monomer to polymerize to form a polymer film.

66. The method of claim 65, wherein said stabilizer is present in said polymer film in an amount effective to promote wound healing.
